(19) United States
(12) Plant Patent Application Publication
(10) Pub. No.: US 2017/0290230 P1
(43) Pub. Date: Oct. 5, 2017

(54) Verbena plant named 'KLEVP15481'

(50) ;

(71) Applicant: Klemm+Sohn GmbH & Co. KG, Stuttgart (DE)

(72) Inventor: Ruijun Li, North Parramatta (AU)

(73) Assignee: Klemm+Sohn GmbH & Co. KG, Stuttgart (DE)

(21) Appl. No.: 14/999,107

(22) Filed: Mar. 30, 2016

(51) Int. Cl. A01H 5/02

(52) U.S. Cl. CPC A01H 5/02
USPC PLT/308

(57) ABSTRACT

A new Verbena plant particularly distinguished by large pink flowers with large white eyes, and a semi-upright growth habit, is disclosed.

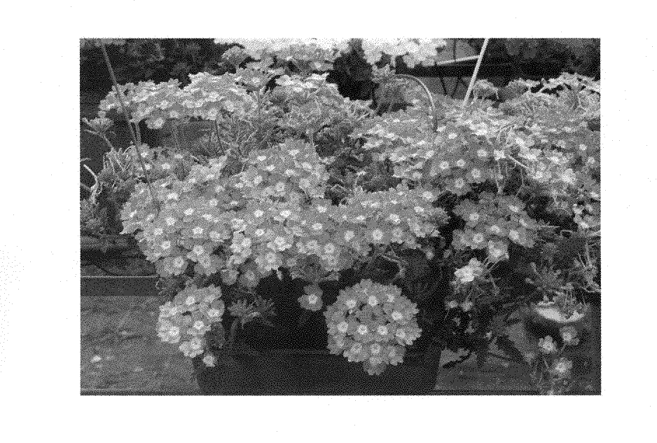

Genus and species: Verbena×hybrida.

Variety denomination:

‘KLEVP15481’.

### BACKGROUND OF THE NEW PLANT

The present invention comprises a new and distinct variety of Verbena, botanically known as Verbena×hybrida, and hereinafter referred to by the variety name ‘KLEVP15481’. ‘KLEVP15481’ originated from a cross-pollination conducted in January 2011 in Cobbitty, New South Wales, Australia between the proprietary female Verbena variety ‘V9113’ (unpatented), and the proprietary male Verbena variety ‘V974’ (unpatented).

The seeds from the cross were sown and plants were grown for evaluation, where an individual plant designated ‘KLEVP15481’ was selected from the group of plants in Cobbitty, New South Wales, Australia in October 2011.

In October 2011, ‘KLEVP15481’ was first vegetatively propagated by vegetative stem cuttings in Cobbitty, New South Wales, Australia. ‘KLEVP15481’ was found to reproduce true to type in successive generations of asexual propagation via vegetative stem cuttings.

### SUMMARY

The following are the most outstanding and distinguishing characteristics of this new variety when grown under normal horticultural practices in Cobbitty, New South Wales, Australia.

1. Large pink flowers with large white eyes; and

2. A semi-upright growth habit.

### DESCRIPTION OF THE PHOTOGRAPH

This new Verbena plant is illustrated by the accompanying photograph which shows the plant's overall plant habit including form, foliage, and flowers.

The photograph is of a 13-week-old plant grown in a greenhouse in Stuttgart, Germany in July 2013. The colors shown are as true as can be reasonably obtained by conventional photographic procedures.

### DESCRIPTION OF THE NEW VARIETY

The following detailed descriptions set forth the distinctive characteristics of ‘KLEVP15481’. The data which define these characteristics were collected from asexual reproductions carried out in Cobbitty, New South Wales, Australia. Data was collected on three-month-old plants grown in a greenhouse in Cobbitty, New South Wales, Australia in June 2015 in Cobbitty, New South Wales, Australia. Color references are to The R.H.S. Colour Chart of The Royal Horticultural Society of London (R.H.S.) 2007.
Classification:
   Family.—Verbenaceae.
   Botanical.—Verbena×hybrida.
   Common.—Verbena.
   Designation.—‘KLEVP15481’.
Parentage:
   Female parent.—The proprietary female verbena variety ‘V9113’ (unpatented)
   Male parent.—The proprietary male verbena variety ‘V974’ (unpatented)
Plant:
   Growth habit.—Semi-upright.
   Height (from top of soil).—36.0 cm.
   Width (horizontal plant diameter).—41.0 cm.
   Time to produce a finished flowering plant.—Approximately 8 weeks.
   Time to initiate and develop roots.—3 weeks.
   Root description.—RHS 155B (White); fine and fibrous roots.
Stems:
   Number of branches per plant.—Around 20.
   Length.—26.0 cm.
   Diameter.—0.26 cm.
   Internode length.—Approximately 2.5 cm.
   Color.—RHS 130D.
   Anthocyanin.—Absent.
   Texture and appearance.—Pubescent, round.
Leaves:
   Arrangement.—Opposite
   Immature and mature leaf color.—Upper surface: RHS 141A Lower surface: RHS 141C.
   Length.—3.5 cm.
   Width.—1.4 cm.
   Shape.—Lanceolate.
   Apex.—Acute.
   Base.—Obtuse.
   Margin.—Entire.
   Texture.—Upper surface: Leathery Lower surface: Rugose
   Venation type.—Pinnate.
   Venation color (both upper and lower surfaces).—RHS 141D.
   Petioles.—Length: 0.3 cm Diameter: 0.1 cm Color: RHS 141D Texture: Leathery.
Flower buds:
   Shape.—Tall, cylindrical.
   Length.—0.5 cm.
   Diameter.—0.2 cm.
   Color (at tight bud).—RHS N74B.
Inflorescence:
   Blooming habit (flowering season).—Continuously.
   Lastingness of individual blooms on the plant.—7 to 10 days.
   Fragrance.—Absent.
   Inflorescence type.—Umbel.
   Length of inflorescence.—4.2 cm.
   Diameter of inflorescence.—5.4 cm.
   Number of florets per inflorescence.—More than 14.
   Peduncle.—Color: RHS 141A Length: 1.1 cm Diameter: 0.2 cm.
Florets:
   Length.—1.6 cm.
   Diameter.—2.2 cm.
   Texture.—Glossy.
   Form.—Single.
   Corolla tube length.—1.3 cm.
   Corolla tube color (outer surface).—RHS 56A.
   Petals.—Length of lobe: 1.4 cm Diameter of lobe: 1.3 cm Lobe shape: Round Apex: Acute Base: Obtuse Margin: Entire Texture (both upper and lower surfaces): Glossy, satiny Color: Immature: Upper surface: RHS 61C Lower surface: RHS 63B Mature: Upper surface: RHS N57C Lower surface: RHS 70C.
   Pedicels.—Not visible.
Sepals:
   Shape.—Lanceolate.
   Apex.—Acute.
   Base.—Obtuse.
   Margin.—Entire.
   Length.—0.45 cm.
   Diameter.—0.36 cm.
   Color.—RHS 141C.
   Texture (both upper and lower surfaces).—Hairy, dull.
Reproductive organs:
   Stamens.—Present Quantity: 4 Color: RHS 139D Filament length: 0.5 cm Filament diameter: 0.1 cm Anther: Shape: Elliptic Color: RHS 150B Length: 0.5 cm Diameter: 0.2 cm Pollen amount: Moderate.
   Pistils.—Number: 1 Length: 1.1 cm Diameter: 1.5 cm Stigma: Color: RHS 144A Appearance: Ovate Length: 0.1 cm Diameter: 0.1 cm. Style: Color: RHS 139C Length: 0.9 cm Diameter: 0.1 cm Appearance: Long.
Fruit and seed set: None observed.
Disease and pest/insect resistance: Good tolerance to powdery mildew (Podosphaera xanthii).
Comparison with parental lines and known variety: ‘KLEVP15481’ is distinguished from its parents as described in Table 1.

TABLE 1
Comparison with Parental Lines

| | | Female parent | Male parent |
|---|---|---|---|
| Characteristic | ‘KLEVP15481’ | ‘V9113’ | ‘V974’ |
| Flower color | Pink | Red | Pink |
| Flower size | Larger than | Smaller than | Smaller than |
| | ‘V9113’ and | ‘KLEVP15481’ | ‘KLEVP15481’ |
| | ‘V974’ | | |

‘KLEVP15481’ is most similar to the commercial verbena ‘Balazwilro’, also known commercially as ‘Aztec Wild Rose’ (U.S. Plant Pat. No. 17,101). Differences between the varieties are described in Table 2.

TABLE 2
Comparison with Similar Variety

| Characteristic | ‘KLEVP15481’ | ‘Balazwilro’ |
|---|---|---|
| Appearance of eyes | Large, white eyes | Small, white eyes |
| Immediate color | RHS 76B (Light-violet) | None |
| bordering eyes | | |

I claim:

1. A new and distinct variety of Verbena plant designated ‘KLEVP15481’ as illustrated and described herein.
